(19) United States
(12) Patent Application Publication
(10) Pub. No.: US 2015/0283313 A1
(43) Pub. Date: Oct. 8, 2015

(54) COIL FOR INDUCTIVE TRANSCUTANEOUS ENERGY AND/OR DATA TRANSFER FOR ACTIVE MEDICAL IMPLANTS

(71) Applicant: EM-TEC GMBH, Finning (DE)

(72) Inventor: Elmar Huber, Kaufering (DE)

(73) Assignee: em-tec GmbH, Finning (DE)

(21) Appl. No.: 14/435,856

(22) PCT Filed: Oct. 16, 2013
(86) PCT No.: PCT/DE2013/000601

§ 371 Date: Apr. 15, 2015

(30) Foreign Application Priority Data
Oct. 16, 2012 (DE) 20 2012 009 867.5

(51) Int. Cl. A61M 1/12

(52) U.S. Cl. CPC A61M 1/127; A61M 2207/00

(57) ABSTRACT

The invention relates to a coil for inductive transcutaneous transfer of electrical power for the power supply of active medical implants, where coil windings are designed with a conductor and run around a centre in spiral shape.

The invention relates to a coil for inductive transcutaneous energy and/or data transfer for active medical implants, where coil windings are designed with a conductor and run around a centre in spiral shape.

Numerous coils for inductive transcutaneous energy supply of active medical implants are known from the prior art.

Transcutaneous wireless energy supply to medical implants (such as blood pumps, cardiac support systems or artificial hearts) is primarily accomplished by way of magnetic induction by means of at least one primary coil (outside the body) through which an alternating current flow and at least one secondary coil (inside the body) through which an alternating current also flows as a result of the induction effect of the magnetic alternating current produced by the primary coil. The coils used for this inductive transcutaneous energy transfer usually consist of spirally wound, electrically insulated copper wires or copper litz wires in which the conductors (wires or litz wires) of one winding turn each lie parallel to the conductors of the neighbouring turns. In order to achieve the highest possible inductances, the turns lie close to one another and in order to achieve the best possible removal of the heat produced by the Ohmic wire losses, these coils are always designed to be oblate to the winding axis (e.g. WO 2009/029977 A1, in particular FIG. 18 there).

Specifically in the case of systems designed for high powers in the range of up to about 50 Watts such as, for example, for the supply of blood pumps, cardiac support systems or artificial hearts, high currents occur on the primary and on the secondary side which on the one hand cause the already mentioned dissipated heat and on the other hand are responsible for the noticeable occurrence of the so-called proximity effect.

The term proximity effect is understood as the current constriction in very closely adjacent conductors through which alternating currents flow. The current constriction is caused by the mutual induction of eddy currents.

Thus, in the usual coils for inductive transcutaneous energy transfer according to the prior art, particularly in the case of high transferred powers, the following problem arises: on the one hand the coils should be designed to be as oblate as possible in order to achieve the highest possible inductance and the best possible heat dissipation within the given scope. With the usual coil structure this has the result that the conductors of all the windings should lie as close as possible to their neighbouring windings as a result of the parallel winding guidance over the entire conductor length.

Consequently, on the other hand a very defined proximity effect occurs which in turn due to the current constriction leads to an increased effective conduction resistance and as a result to increased dissipated heat.

A further problem with conventional oblate, tightly spirally wound coils for inductive transcutaneous transfer of high powers is the fixing of the conductor ends, i.e. the part of the conductor (wire or litz wire) wound to form the coil which must remain in the inside and at the edge of the coil for the purpose of electrical connection to other electronic components. Usually these conductor ends are connected to the coil by adhesion or mechanical aids so that an undesired unwinding of the coil is prevented.

In addition, the usual oblate, tightly spirally wound coils for inductive transcutaneous transfer of high powers are very rigid with respect to bending perpendicular to the winding plane and as a result not optimal for an application as an implant which should be adapted as well as possible to a body location.

It is the object of the invention to improve these coils known from the prior art.

This object is solved according to a first aspect of the invention by a coil for inductive transcutaneous energy and/or data transfer for active medical implants, where coil windings are designed with a conductor and run in spiral shape around a centre, where n points of intersection exist between two neighbouring complete windings, which define a winding plane wherein n is an odd number >3 and the position of the wound conductor with regard to the winding plane before and after the point of intersection relative to the winding plane alternates between a low position below the winding plane and a high position above the winding plane, and the coil has a sheath of a biocompatible material.

Since n is an odd number, this is equivalent to the circumstance that the states of two adjacent windings between their common points of intersection alternate radially between a low position and a high position.

With such a coil it is possible to provide a coil suitable for the transcutaneous transfer of high power which for a given diameter and small extension along the winding axis (i.e. retaining the oblate design as far as possible) has the smallest possible proximity effect and at the same time enables a very good removal of lost heat, makes it possible to fix the conductor ends in a simple manner due to their special design and which is flexible with respect to bending perpendicular to the winding plane and thus can be adapted particularly well to the body site at which it is to be implanted.

It is advantageous if the coil has an idle-running coil quality of 200-250. A high coil quality Q is a prerequisite for high efficiency with simultaneously low heat evolution at high powers. The coil can be operated in a frequency range of 50-250 kilohertz, preferably in the range of 100-200 kilohertz.

Alternatively or cumulatively to a transcutaneous energy transfer, a data transfer via the coils themselves, in particular a unidirectional data transfer is possible. Alternatively or cumulatively a separate telemetric unit can be provided for bidirectional and/or unidirectional data transfer.

In addition, the spiral shape can only begin at a certain distance from the centre.

It is advantageous if radially adjacent points of intersection always enclose the same angle in relation to the centre. These points of intersection therefore lie on a straight line which leads into the coil centre.

It is further advantageous if the winding plane is a curved plane. It is thus possible to adapt the implant to a specific body location. This enhances the comfort of the implant wearer.

It is further advantageous if at a position at a greatest distance of the conductor from the winding plane, a conductor outer side has a distance of at least 0.5, in particular of at least 1.0 of the lead diameter to the winding plane. Since the conductor in a certain turn only has contact with itself in the two adjacent turns at the points of intersection, due to the substantial reduction in the magnetic interaction between the windings where the distance between its surfaces is designated with d, the proximity effect is reduced significantly, approximately according to a d⁻³ dependence.

The low position and high position with respect to the winding plane can in particular lie in the region of the radius of the conductor 1 as a result of the substantial reduction in the magnetic interaction so that the largely oblate shape of the coil advantageous for heat removal can be retained. Heat measurements and thermography measurements have shown that with suitably wound coils, due to the reduced proximity effect, the production of lost heat is smaller and the removal of lost heat is more efficient than in comparable conventional coils. Smaller distances are already advantageous, even greater distances are feasible.

As a result of the alternating between the low and the high position, cavities can be formed which run continuously radially outwards from the coil centre. These enable various uses of these cavities.

In particular one conductor end can be guided through a cavity. It is therefore unnecessary to fix this conductor end by adhesions or mechanical aids. In addition, the end can be guided elegantly in the cavity.

It is therefore particularly advantageous if an inner conductor end is guided outwards through a cavity. This contributes to a flat configuration of the coil since the inner conductor end can be guided in the cavity.

It is further advantageous if a sensor, in particular a temperature sensor, is guided through a cavity. This can be inserted and also woven in. A plurality of sensors can also be provided. These can be used for temperature and power monitoring. In particular temperature sensors can especially comprise thermistors or Hall probes.

A use of the cavity for insertion of an antenna again both in particular by insertion and also weaving in is also advantageous. An antenna can thus be guided in the cavity. In particular, this can comprise an antenna for communication, for example, a vertically placed frame antenna for bidirectional communication.

Furthermore, a support element can be guided in a cavity. The cavity can thus be used to introduce rigid or flexible support elements such as support holders, pins, rods, tubes. These can possibly be equipped with guide grooves. They serve as spacers, carriers and as litz wire guidance. They can be made of non-magnetic metals, in particular of aluminium or of thermally conductive plastic or ceramic for improvement of the thermal heat compensation in the sense of a thermal homogenisation and of temperature-resistant insulating plastics. Combinations of the materials are also possible.

It is further advantageous if a support element is curved. Concave, convex or differently shaped coil variants can be produced with curved or differently shaped support elements, in particular support holders in the form of pins or carriers. Pre-formed coils can be better adapted to a body contour. By introducing one or more objects as stated, for example, in the form of straight or curved rods into one or more cavities, a defined deformation of the coil with respect to the winding plane can be produced. In particular, the shaping objects can each have a specific curvature. This curvature can differ from shaping object to shaping object. As a result of suitable design and type of objects, the coil can furthermore have a certain flexibility with respect to bending and perpendicular to the winding plane. By introducing the different elements into these cavities or weaving the elements with the windings which delimit these cavities, the various elements can be efficiently introduced without being secured by an additional fixing device. If the elements comprise conductor ends, a desired unwinding of the coil is thus effectively prevented.

It is further advantageous if two conductor ends are twisted at the coil connection. Thus, undesired scattering fields are minimised.

One winding already has a positive effect here. However, numerous windings are also possible. It is further advantageous if the spiral shape as outer contour has an oval shape, in particular a Lémesch oval, i.e. the shape of a plaster or an elliptical shape. It is further advantageous if the coil has a plurality of winding planes. This is the case, for example, with a honeycomb coil. In particular three or more planes are feasible.

The points of intersections can be firmly connected to one another. This is particularly flexible. Since a contact only exists at the points of intersection, the friction of the conductor surface between the windings is reduced. This leads to an increased flexibility of the coil with respect to bending perpendicular to the plane of action.

However, the stiffness of the coil can be influenced by a connection of the points of intersection. This can be accomplished, for example, by binding, for example, with a thread, gluing or also by suitable resins or varnishes. A flexible coil can, for example, be glued only at the central points of intersection such as, for example, the last winding. Thus, all non-connected coils can easily be displaced with respect to one another, which gives the coil a more flexible, softer property. If a stiffer or rigid coil is desired, a rigid structure is obtained if all points of intersection are connected to one another, or the entire coil is dipped in suitable varnish or adhesive. Two-component adhesive, for example, is suitable as adhesive. Here also the stiffness can be influenced by the mixing ratio of the hardener. Since cyanoacrylate adhesives as used in secondary adhesives can react strongly to moisture such as water, these are not suitable for coils which are to be siliconised subsequently. In particular, biocompatible adhesives having a USP class VI licencing, such as for example the silicone adhesive Silupran 4200 or a two-stage siliconisation process come into consideration. Very elastic connections can thus be produced.

It is advantageous if the biocompatible material for the sheath is a silicone. In order to enable an implantation, the coil with biocompatible material must be embedded hermetically tightly in a sheath. It is advantageous if the coil is enclosed directly in a corresponding sheath.

The biocompatible material can also be another long-term tested biocompatible plastic such as polymers such as polyurethane (PUR), PTFE, PEEK or another temperature-resistant thermoplastic. In the USP Class VI (Implantation Tests according to ISO 10993-6, intracutaneous test, toxicological and cytotoxic test according to ISO 10993-5, haemocompatability according to ISO 10993-4) the following materials, in particular the following polymers are mentioned as reference materials in accordance with ISO10933-12: PTFE polytetrafluoroethylene (Teflon) which is sterilisable, in particular using an autoclave, FEP fluoroethylene propylene which is also sterilisable in particular with ETO and autoclave, ePTFE which is implantable, which supports endothelisation and is antithrombogenic, PFA perfluoroalkoxy as well as ETFE ethylene tetrafluoroethylene.

In this USP class systemic injection, an intracutaneous test and an implantation test are performed. Mixtures of these materials as well as various layers and sheaths are feasible.

Advantageously the biocompatible material has a Shore hardness A of 20 to 50, preferably of 20 to 40. As a result of the Shore hardness it is possible to vary the flexibility of the implant. A softer implant has the advantage that an increased flexibility, e.g. as response to body movements is provided. This is particularly advantageous for implanted coils. In the case of extracorporeal coils, higher Shore hardness can also be appropriate.

It is further advantageous if the coil with sheath has a thickness of 10 mm, preferably of 5-8 mm. The thinner the coil, the more pleasant it is to wear as an implant. Greater thicknesses are indeed feasible, but this substantially reduces the comfort.

It is further advantageous if the sheath comprises a fastening apparatus. It is thus possible to fasten the implant in the body. Here the fastening apparatus can be an eye or a sterile band. An eye is used during implantation of the coil for surgical fixing of the sheath to secure against undesirable migration in the body by sewing with solid biological tissue. If this fastening aid is not required as a result of the anatomical position, with an appropriate choice of sheath material this can easily be removed by the surgeon with a scalpel cut. A constructive alternative to this is to mould a sterile band, for example, in the form of a circumferential ring.

The coil can have a ferromagnetic plate. This can be moulded into the sheath. It is used for shielding. Here care will be taken to ensure that the ferromagnetic plate preferably lies in the order of magnitude of 0.2 to 2 mm, in order not to increase the overall thickness of the implant unnecessarily.

It is advantageous if the coil has two taps, one or no taps.

It is further advantageous if the conductor is an HF litz wire. High-frequency litz wires (HF litz wires) consist of individual insulated wires which are woven into thin bundles with a typical diameter of 0.5 mm-3 mm. These are already used to reduce losses as a result of, for example, the skin and proximity effect. Copper litz wires, for example, are possible. Alternatively silver litz wires can also be used, these having an increased electrical and thermal conductivity and a better biocompatibility. Here a litz wire can typically comprise one hundred to two hundred and fifty wires each having a diameter of 0.05 to 0.1 mm. These can, for example, be spun around with polyurethane (so-called enamelled-insulated copper litz wires) and/or singly or multiply. The wrap spinning can consist of artificial silk, Mylar or other plastic films having good insulation properties. Different bundlings and forms of insulation for HF litz wires can be seen in FIG. 21.

A second aspect of the invention relates to a pair of coils for inductive transcutaneous transfer of power for the energy supply of active medical implants having an implantable secondary coil and an external primary coil, wherein one of the coils, in particular the implantable secondary coil is a coil according to the invention. Such a coil enables transcutaneous energy transfer, optionally data transfer, in particular unidirectional data transfer can be provided.

It is advantageous if the pair of coils has an alignment aid. In order to avoid any displacement of the coil, it is helpful for the user that the TET system delivers a message as to how well the coils are centred, i.e. how good is the inductive coupling and therefore the energy transfer efficient. Here there are at least three possible solutions:

Firstly, relative movements can be minimised by fixing the coils for example by a surgical fixing method to eyes.
Secondly, an alignment can be accomplished by cylindrical disk magnets, preferably in the form of cylindrical ferrite magnets having axial permanent magnetisation, preferably of the quality Y35. Ferrite-based ferrite magnet disks (composites) are a simple solution to assist the patient and physician with the optimal positioning of the coils. The magnetic forces of these typically axially magnetised disk magnets (thus, in particular having a diameter of 20 mm) can be perceived clearly over distances of far more than 10 mm. The additional weight of these magnetic disks is between 5 and 10 grams.
Thirdly, an electronic feedback with navigation aid can be provided. The evaluation and feedback of the received power or the magnitude of the voltage at the secondary coil can be used as electronic feedback.

It is further advantageous if the pair of coils comprises safety electronics. This makes it possible to ensure safe operation of the pair of coils during implantation.

The safety electronics can comprise a foreign object detection and/or an analogue test enquiry and/or a digital identification and/or an overcurrent protection and/or a voltage clamp and/or a temperature detection and/or a high voltage surge stopper and/or an ESD protection and/or switch-off possibility and/or a stand-by mode of the active synchronous rectification.

A foreign object detection (POD) functions as follows: in order to prevent heating by eddy currents taking place in an electrically conductive foreign body during inductive energy transfer, the transmitted power is continuously compared with the received power. If a large difference is obtained, this indicates the presence of a metal foreign body. The energy transfer must then be reduced or switched off. By means of a visual or audible alarm generation, the patient is instructed to remove the foreign body. After removal, the transfer can be continued.

An analogue test enquiry (Analog Ping) should be understood as follows: during every switch-on process of the coil driver, it is checked with a short oscillatory switch-on pulse whether an adequate receiver with predicted secondary coil inductance is provided. To this end, the response caused by the counter-inductance is evaluated. If no response, an error case exists and a corresponding alarm must be issued.

A digital identification (digital Ping) should be understood as follows: in order to be quite sure that no unknown receiver can cause an undesired energy transfer, a digital identification of the receiving coil(s) is performed.

Furthermore, the already-mentioned protection and safety functions can additionally be provided alternatively or cumulatively on the secondary side: overcurrent protection, voltage limitation, temperature detection, high voltage surge stopper, ESD protection, switch-off possibility and standby of the active synchronous rectification.

It is further advantageous if the pair of coils transfers powers in the order of magnitude of 5-50 Watts, in particular 5-40 Watts, in particular 10-30 Watts. This enables a sufficient transfer of energy for active medical implants.

It is particularly advantageous if the pair of coils is suitable for a transmission over a distance of 5-40 mm, in particular of 5-15 mm, in particular of 10-15 mm. These are the distances which normally occur in transcutaneous energy supply, where a short distance is advantageous for operation of the system but a larger possible deviation ensures an increased security for the implant wearer.

A third independent aspect of the invention relates to a coil system comprising a pair of coils according to the invention, which this comprises a further pair of coils. Thus, two independent power pairs are achieved. The coil system can also comprise a second implantable secondary coil, in particular a coil according to the invention. Thus, one current path can be used for a VAD and the other for charging the internal energy storage device.

A fourth aspect of the invention relates to an active medical implant, in particular a cardiac support system, in particular having a power requirement of 5-50 Watt comprising a coil in particular according to the invention or a pair of coils, in particular a pair of coils according to the invention, or a coil system, in particular a coil system according to the invention. This can occur in each case alternatively or cumulatively.

A final aspect of the invention relates to a method for fabricating a coil system.

This can comprise a winding method. This can comprise the following steps:
1) Cutting HF litz wires to the given or calculated length with double allowance for the desired connection length (typically: 5-10 cm)
2) The clean-cut ends are dipped in a lead-free dipping solder bath (depending on the litz wire usually at >=450° C.) to about 10 mm. Wait until the insulation varnish layer is evaporated and the ends are tin-plated (about 5 seconds).
3) Recommendation: DC resistance measurement for quality assurance
4) At one end fixed with adhesive tape on the outer spoke end after the connection length.
5) The HF litz wire is initially guided along one spoke from outside to inside.
6) The winding device is then turned to the right (for a dextrorotary coil) or to the left (for a levorotatory coil). During turning the litz wire is guided at almost constant tension at each spoke alternately upwards or downwards (states). Since the number of spokes is odd, after precisely one revolution the other state is reached (top if beginning from the bottom or conversely).
7) The winding tool is turned manually or driven ever further by a stepper motor in the selected direction of rotation.
8) After the next complete revolution with alternating change of state from spoke to spoke, the initial state is reached.
9) Note: after the second revolution the connecting lead should run automatically in the cavity of the coil. If this is not the case, this should be looped in.
10) Continue in this way until the desired outside diameter of the coil is reached or the litz wire reaches the end.
11) Fixing the end at the desired exit point (usually always after a complete revolution=number of turns).
12) Optional recommendation: insulate a connecting conductor again at the entry point possibly right into the cavity with shrink hose.
13) Optional recommendation: conductors can be twisted to minimise scattering and disturbing influences (typically one stroke per cm is sufficient)
14) Connecting or adhesive bonding of the points of intersection (see description)
15) Spokes can now be removed from the winding tool.
16) After removing the spokes, there is space for insertion of any sensors (e.g. thermistors)

A vertical winding technique is also feasible:

Similarly to the winding technique in the horizontal position described above, the winding plane can also be turned through 90°. This gives, for example, a winding device (FIG. 25) with which short cylindrical air coils can be produced. On account of its height of preferably 10 to 20 mm, these are not very suitable as transcutaneous coils. This winding technique can, on the other hand very well be used as an external primary coil. Advantage: large region penetrated by field, all turns make almost the same contribution to the electric flux density. FIG. 26 shows how shaped coil windings are also possible. A conical coil can be produced with this winding device which could be adapted to any skin bulging.

A winding thus produced can, for example, in the case of a silicone casting process, comprise the following steps:

Biocompatible silicone mixed to the desired Shore hardness is used as casting material. In order to enable a bubble-free casting, the vacuum chamber is degassed. Then an underside is cast in a casting mould, preferably at a height of about one millimetre and hardened for two to three hours in an oven at 30°. Optionally a magnetic disk is placed on the silicone base. The previously fabricated winding is also put in place. The connecting point of the litz wires is sealed with moulding kneading compound. The mould provided with the upper part is filled with silicone. This is followed by degassing in the vacuum chamber. The hardening process can take place overnight for example. Demoulding optionally takes place with the aid of the insertion of compressed air. The mould can then be cleaned with isopropanol.

Apart from the silicone process presented, the coil can also be ensheathed with temperature-resistant thermoplastic elastomers with high-pressure overmoulding [PCT/US2011/030136: WO2011/126791]. The problems accompanying high-pressure overmoulding are disclosed in the same patent specification.

The figure is explained in detail in the following by means of exemplary embodiments with reference to the designation. In the figures:

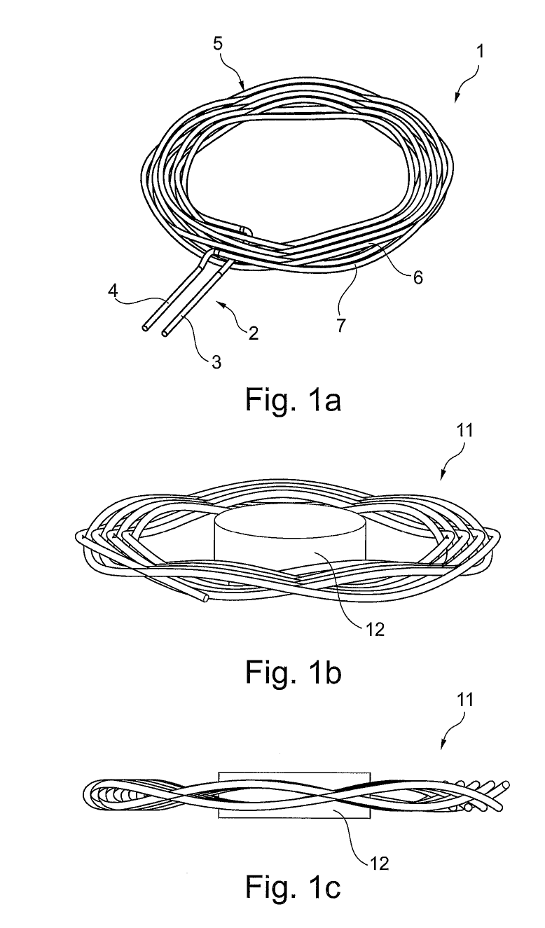

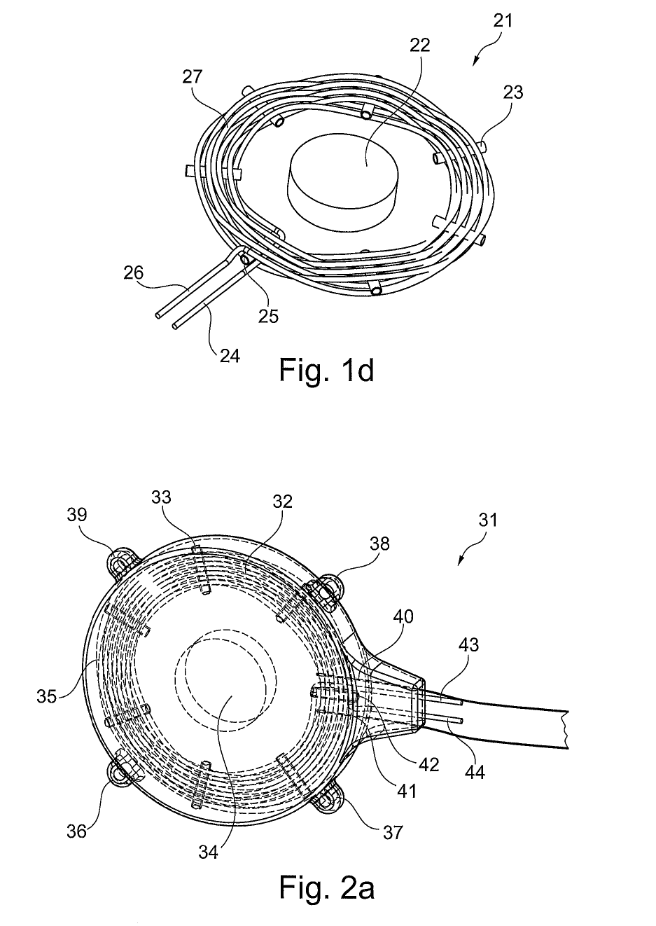

FIG. 1a shows a schematic diagram of a winding for a coil without carrier tube.

FIG. 1b shows a schematic diagram of a winding for a coil with a magnetic disk.

FIG. 1c shows a schematic diagram of a side view of a winding for a coil.

FIG. 1d shows a schematic diagram of a winding for a coil without carrier tube and magnetic disk.

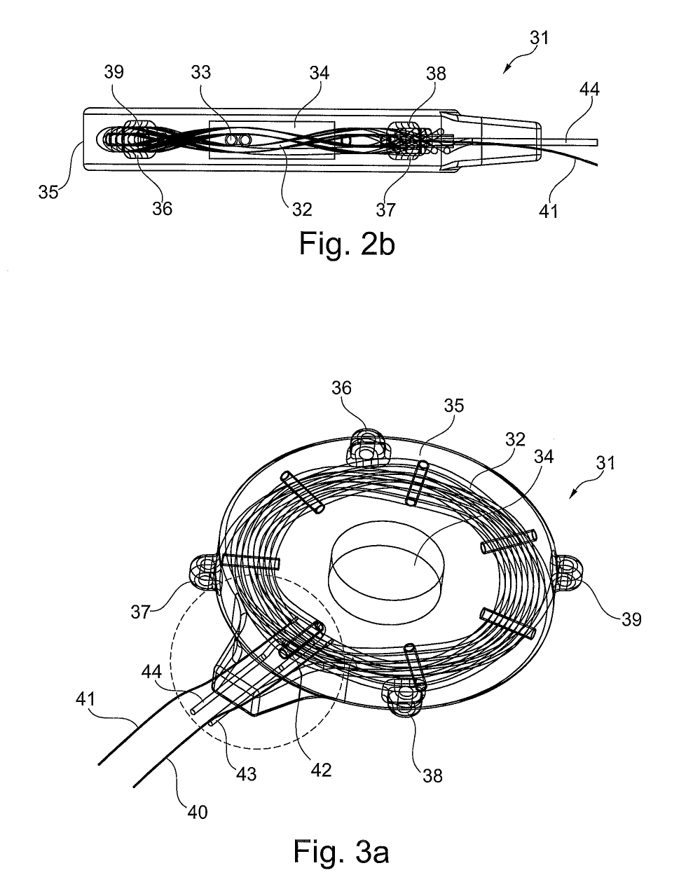

FIG. 2a shows a schematic diagram of a 3D view of a coil embedded in a silicone sheath.

FIG. 2b shows a schematic diagram of a side view of a coil embedded in a silicone sheath.

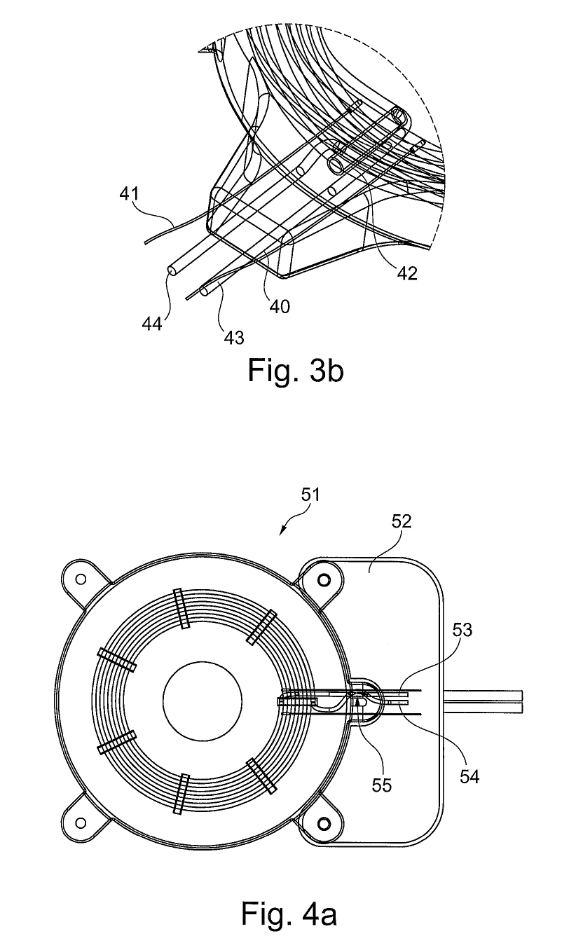

FIG. 3a shows a schematic diagram of a 3D view of a coil with a silicone sheath without driver or receiving electronics.

FIG. 3b shows a schematic diagram of a section showing the connection point of the coil with silicone sheath shown in FIG. 3.

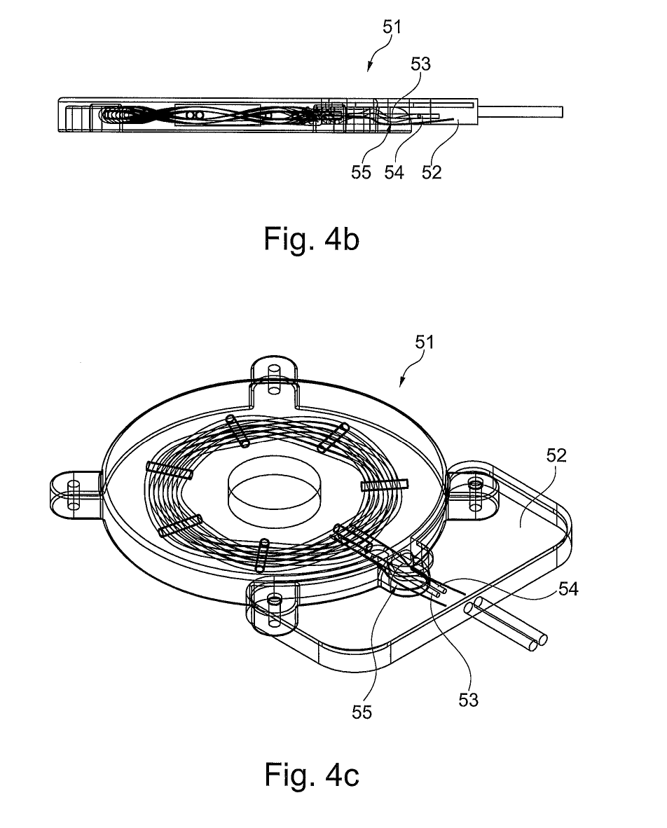

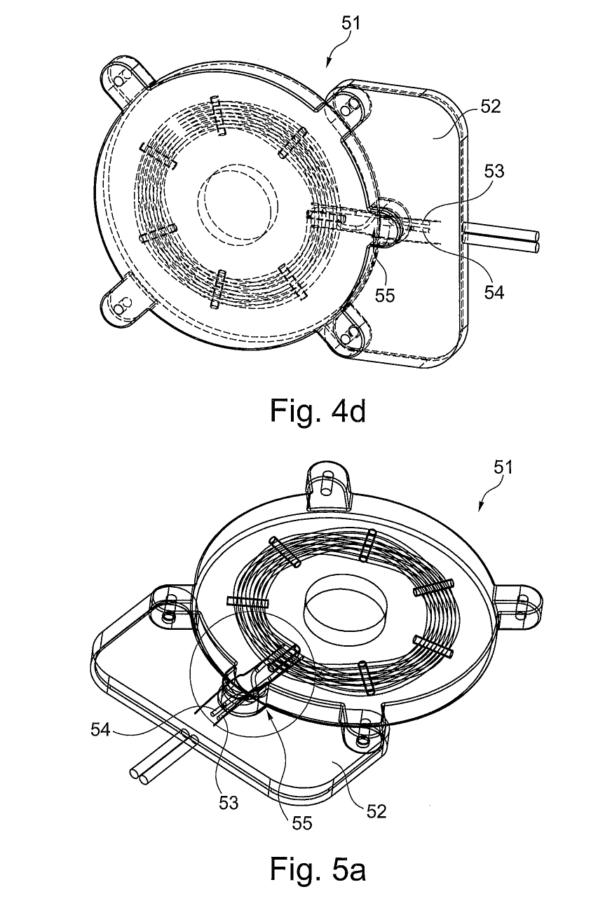

FIG. 4a shows a schematic diagram of a plan view of an individual coil with driver or receiving electronics.

FIG. 4b shows a schematic diagram of a side view of an individual coil with driver or receiving electronics.

FIG. 4c shows a schematic diagram of a 3D view of an individual coil with driver or receiving electronics.

FIG. 4d shows another schematic diagram of a 3D view of an individual coil with driver or receiving electronics.

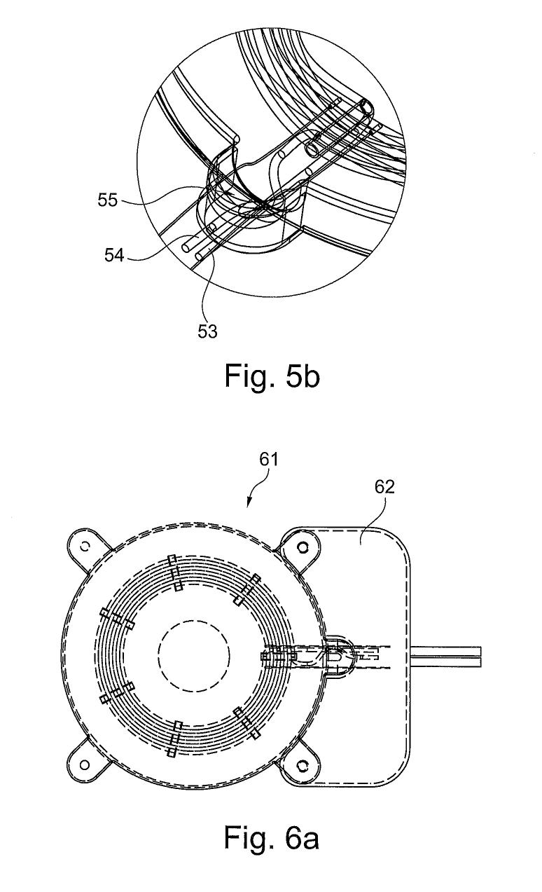

FIG. 5a shows a schematic diagram of a 3D view of an individual coil with driver or receiving electronics.

FIG. 5d shows a schematic diagram of a section showing the connection point to the individual coil shown in FIG. 5a with driver or receiving electronics.

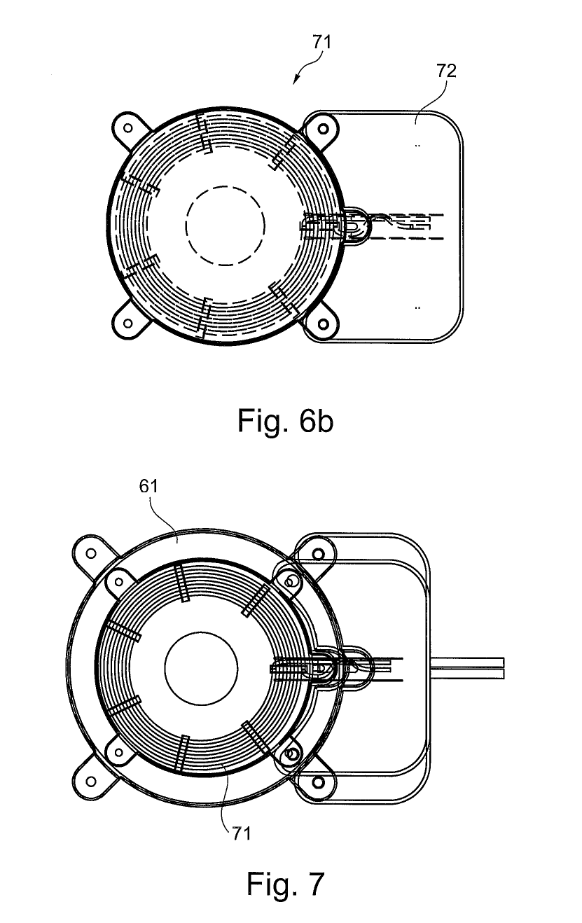

FIG. 6a shows a schematic diagram of a plan view of a larger external primary coil with driver electronics.

FIG. 6b shows a schematic diagram of a plan view of a small implantable coil with the same electronics.

FIG. 7 shows a schematic diagram of a plan view of a coil system.

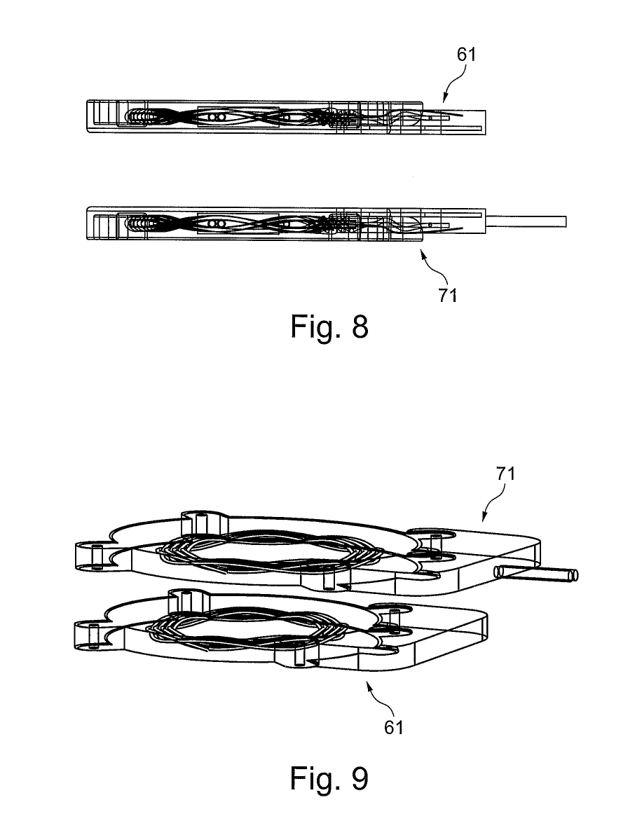

FIG. 8 shows a schematic diagram of a side view of a coil system.

FIG. 9 shows a schematic diagram of a 3D view of a coil system.

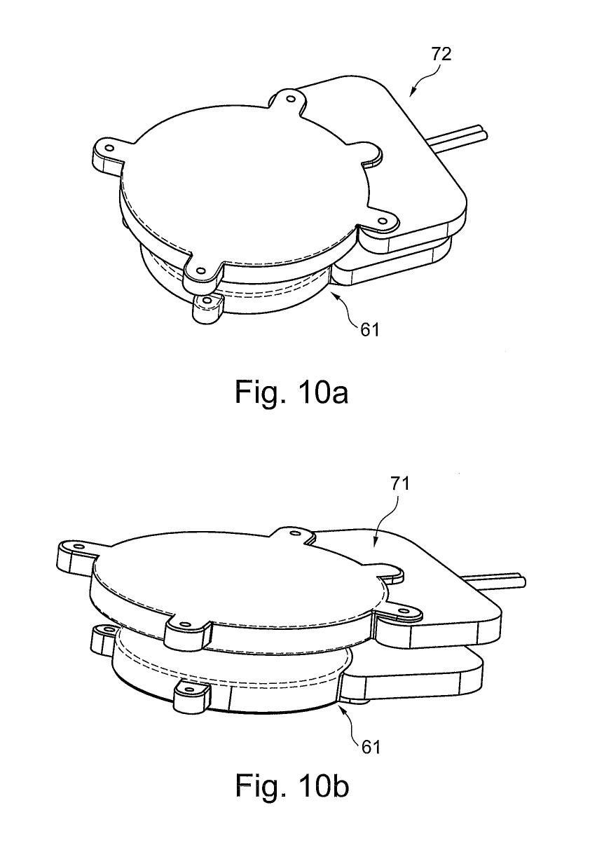

FIG. 10a shows a schematic diagram of a 3D view of the outside view of a coil system.

FIG. 10b shows another schematic diagram of the 3D view of the outside view of a coil system.

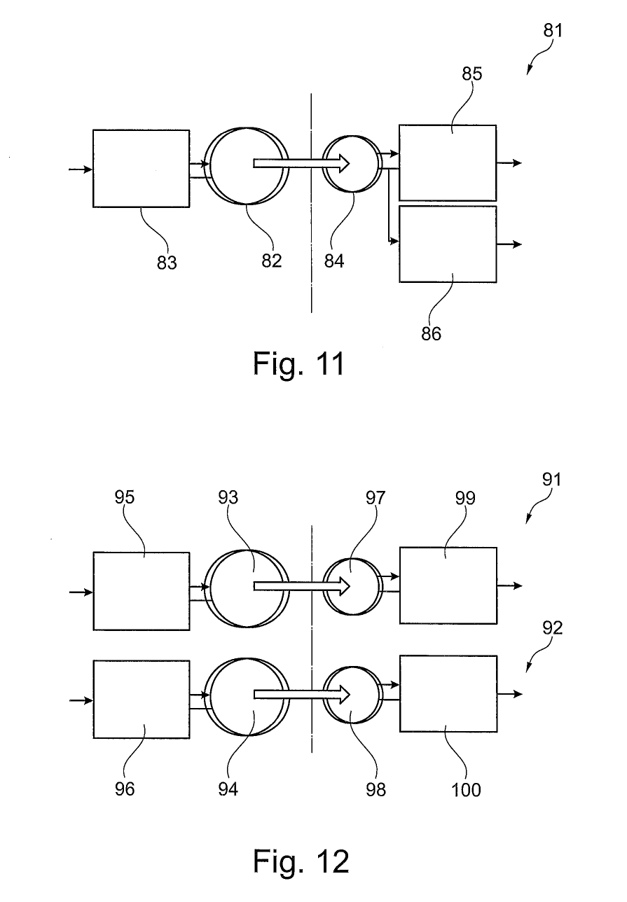

FIG. 11 shows a schematic view of a coil system with a primary coil with driver and a secondary coil which supplies two consumers.

FIG. 12 shows a schematic diagram of a coil system with two independent power paths.

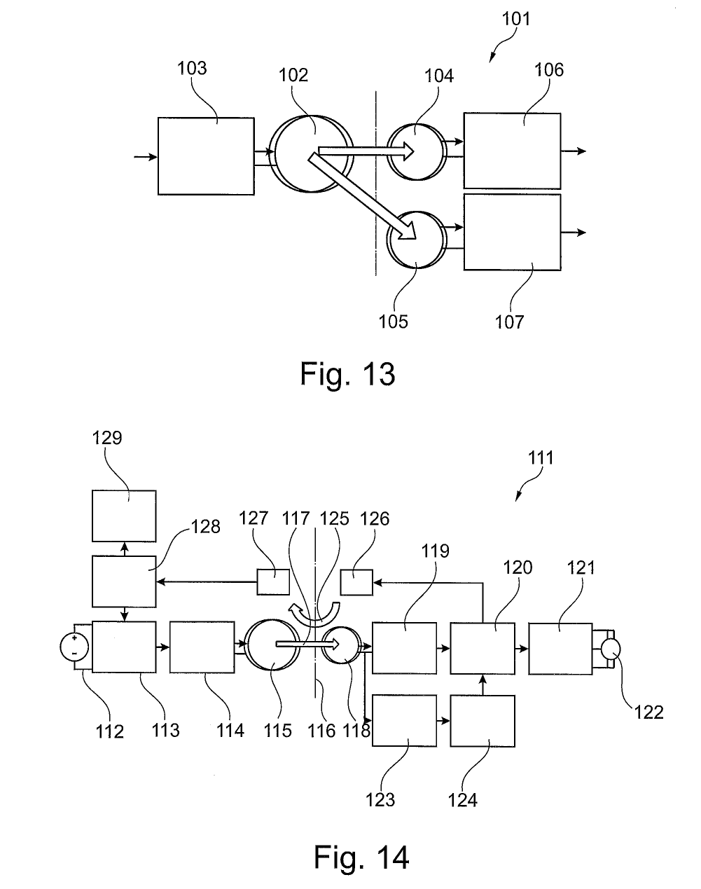

FIG. 13 shows a schematic diagram of a coil system in which a primary coil drives two secondary coils.

FIG. 14 shows a schematic diagram of a transcutaneous energy transfer system.

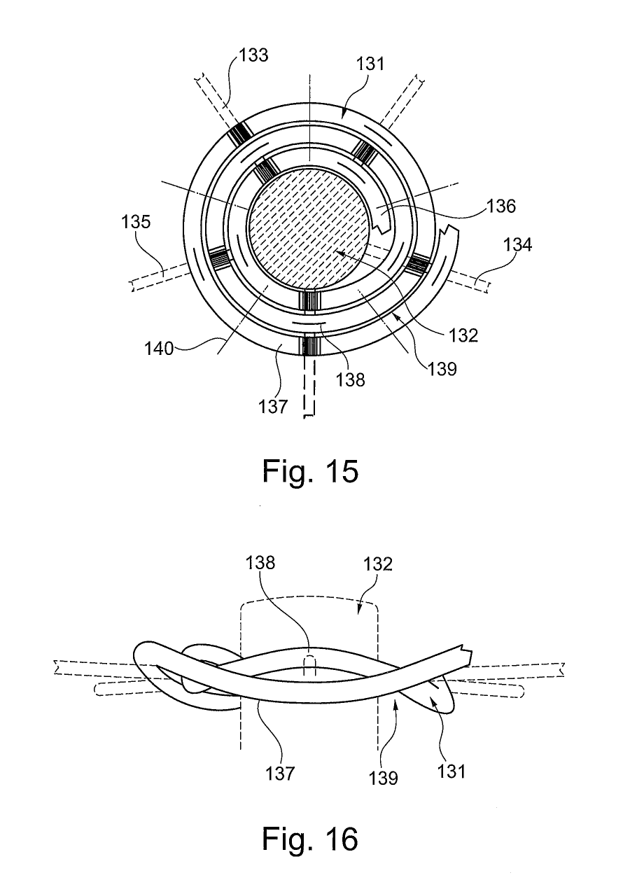

FIG. 15 shows a schematic diagram of a winding device with litz wire in plan view.

FIG. 16 shows a schematic diagram of a winding device with litz wire in side view.

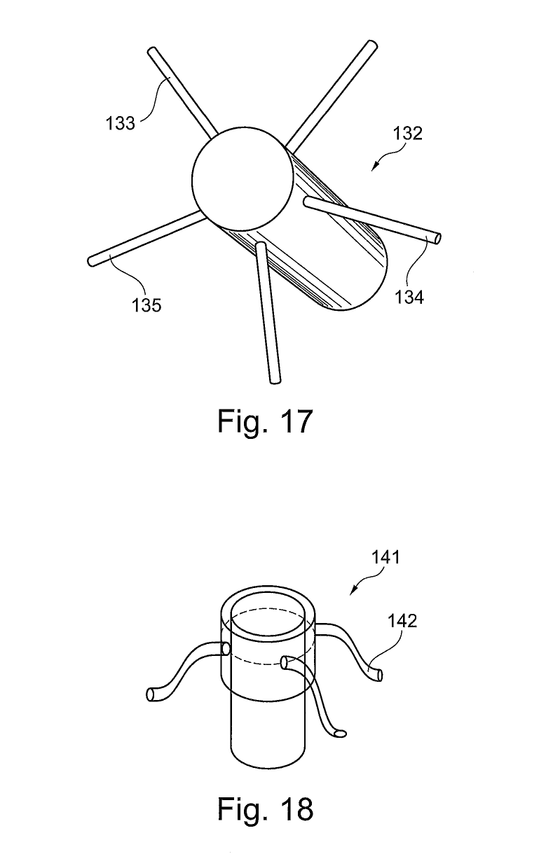

FIG. 17 shows a schematic diagram of a winding device with horizontal pins.

FIG. 18 shows a schematic diagram of a winding device with curved spokes and changeover attachment for enlarging the inside diameter of the coil.

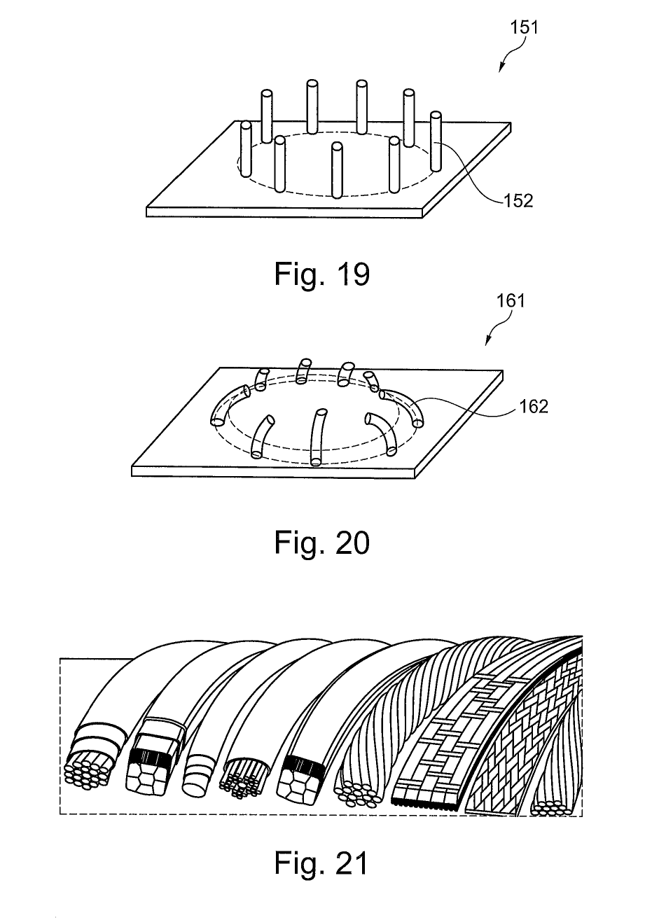

FIG. 19 shows a schematic diagram of a winding device with vertical support points.

FIG. 20 shows a schematic diagram of a winding device with vertical support points where these are moulded.

FIG. 21 shows a schematic diagram of various HF litz wires.

The winding (1) in FIG. 1 is formed from the conductor (2). This has an inner conductor end (3) with which the winding (1) begins on the inner side and is then continued to the outer side, where the conductor ends in an outer end (4). The desired structure is obtained as a result of the alternating winding of the conductor (2) at the points of intersection, such as for example point of intersection (5). The points of intersection, such as the point of intersection (5) in this case form a winding plane. In the present case the winding (1) comprises a winding with seven points of intersection. In the winding in FIG. 1a no carrier tube can be provided. This can, for example, comprise an adhesively bonded variant. FIGS. 1b and 1c shows a winding (11). In addition to this, a magnet disk (12) is shown. This forms a part of an alignment aid (other parts are not shown).

In addition, along with the magnet disk (22) retaining tubes such as, for example, (23) can be provided for the winding (21) as shown in FIG. 1d. The inner conductor (24) is also guided through one of the openings in which a retaining tube, here (25) is inserted. The outer conductor (26) and the inner conductor (24) are not twisted. The winding has seven points of intersection such as points of intersection (27) with interposed openings into which the retaining tubes such as, for example, the retaining tube (23) and the retaining tube (25) are inserted.

In a coil (31) as shown in FIGS. 2a, 2b and 3a and in the detailed view 3b, the winding (32) with the carrier tube (33) and the magnet (34) is enclosed by a silicone sheath (35). This has the eyes (36), (37), (38) and (39). In addition, two sensors (40) and (41) are embedded which are also guided through an opening in the winding. A fastening of the coil (31) during implantation is possible through the eyes (36), (37), (38) and (39). The introduction of the sensors (40) and (41) into the corresponding opening of the winding (32) together with the carrier tube (42) as well as the inner conductor end (43) and the outer conductor end (44) into the opening can be seen particularly clearly in the detailed view 3a of the connecting region (45).

The single coil (51) in FIGS. 4a, b, c, d and FIG. 5a and b is provided with a driver or receiving electronics (52). The conductor ends 53 and 54 are twisted before entry into the driver or receiver electronics (52) at the point (55). This is particularly identifiable in the detailed view in FIG. 5b.

A pair of coils as shown in FIGS. 7, 8, 9 and 10a and b is composed of a large external primary coil (61) with driver electronics (62) and a small implantable coil (71) with receiving electronics (72).

In the coil system (81) (FIG. 11), a primary coil (82) with driver (83) supplies a secondary coil (84) which for its part supplies two consumers (85) and (86).

Alternatively two power paths (91) and (92) can be provided in which respectively one primary coil (93, 94) with driver electronics (95, 96) supplies a secondary coil (97, 98) with corresponding consumer (99, 100) (FIG. 12).

Finally however it is also possible to drive two secondary coils (104, 105) in a coil system (101) with a primary driver (102) and a driver (103, which then for their part supply two consumers (106, 107) (FIG. 13). One power path can then be used for a VAD, the other for charging the internal energy storage device.

A coil system with an active medical implant (111) is shown in FIG. 14. In such a system energy is fed into the system (111) by a voltage source (112). The voltage source (112) can comprise a DC voltage source in the form of a battery, a rechargeable battery or a DC power supply. The primary coil (115) is operated via a rectifier (113) and a primary compensation network (114), preferably in the form of a circuit with series or parallel capacitance. The rectifier (113) comprises a switched current driver for the primary coil, typically a class D or E power amplifier (switch-mode coil driver Class D or E). Across the skin barrier (116) energy, here symbolised by the arrow (117), is transferred to the secondary coil (118). This relays the energy via a secondary compensation network (119), for example a circuit having series or parallel capacitance, a rectifier (120) with voltage limiter and/or filter and/or safety electronics or control electronics and a control unit (121) to the consumers (122). The consumer can comprise a cardiac support system (VAD), artificial heart (THD) or other active implants having a power requirement of 5-50 Watt, with or without sensors. Typically such a consumer comprises a three-phase brushless DC motor.

At the same time, a circuit is provided for non-contact charging consisting of an internal energy storage device (123) and a charging unit (124). Furthermore, a contactless communication and regulating circuit which enables feedback is provided. In this circuit, data symbolised by the arrow (125) is transferred from a data transmitter (126), for example, via AM or FM modulation to the inductive coupling or directly to a data receiver (127) and demodulated there. An extracorporeal control unit (128) then acts accordingly again on the rectifier. External access is possible through the user interface (129) with visual and/or audible display which can also provide an alarm facility.

FIGS. 15 and 16 show a winding (131) on an auxiliary body (132). The auxiliary body (132), as clearly visible in FIG. 17, here comprises five cylindrical spokes such as the spokes (133, 134, 135). These can be anchored detachably, for example, plugged or screwed in the auxiliary body. If we now begin at this point inside on the auxiliary body (132) and guide a conductor (136) alternatively above and below the spokes such as, for example (133, 134, 135) of the auxiliary body (132), these are located in a lower position (137) and in a higher position (138) with respect to a plane formed by the points of intersection such as, for example, point of intersection (139) which also forms the image plane in FIG. 15. The sequence of positions alternates as a result of the odd number of spokes (133, 134, 135) of the auxiliary body (132) both along the conductor (136) and also radially from one winding row to the next. The contact points between two adjacent windings form the transition between the states in the winding direction of the conductor and lie in the radial direction on straight lines such as (140).

In order to achieve different shapings, a winding device (141) can have curved spokes such as (152). An interchangeable attachment (153) enables the inside diameter of a coil (not shown) to be enlarged (FIG. 18).

A winding can also be produced on a winding device (151) having vertical support points such as, for example, (152) (FIG. 19).

Such support points such as, for example, (172) can also be curbed in a winding device (171) (FIG. 20).

1. Coil for inductive transcutaneous energy and/or data transfer for active medical implants, wherein coil windings are designed with a conductor and run in spiral shape around a center, wherein n points of intersection exist between two neighbouring complete windings, which define a winding plane wherein n is an odd number >3 and the position of the wound conductor with regard to the winding plane before and after the point of intersection relative to the winding plane alternates between a low position below the winding plane and a high position above the winding plane and the coil has a sheath of a biocompatible material.

2. The coil according to claim 1, wherein radially adjacent points of intersection always enclose the same angle in relation to the center.

3. The coil according to claim 1, wherein the winding plane is a curved plane.

4. The coil according to claim 1, wherein at a position at a greatest distance of the conductor from the winding plane, a conductor outer side has a distance of at least 0.5, in particular of at least 1.0 of the lead diameter, to the winding plane.

5. The coil according to claim 1, wherein as a result of the alternating between the low and the high position, cavities are formed which run continuously radially outwards from the coil center.

6. The coil according to claim 5, wherein one conductor end is guided through a cavity.

7. The coil according to claim 6, wherein an inner conductor end is guided outwards through a cavity.

8. The coil according to claim 5, wherein a sensor, in particular a temperature sensor, is guided through a cavity.

9. The coil according to claim 5, wherein an antenna is guided through a cavity.

10. The coil according to claim 5, wherein a support element is guided through a cavity.

11. The coil according to claim 10, wherein the support element is curved.

12. The coil according to claim 1, wherein two conductor ends are twisted at the coil connection.

13. The coil according to claim 1, wherein the spiral shape as outer contour has an oval shape, in particular a Lémesch oval or an elliptical shape.

14. The coil according to claim 1, wherein the coil has a plurality of winding planes.

15. The coil according to claim 1, wherein the points of intersections are firmly connected to one another.

16. The coil according to claim 1, wherein the biocompatible material is a silicone.

17. The coil according to claim 1, wherein the biocompatible material is another long-term tested biocompatible plastic such as, for example, polymer such as polyurethane, PTFE, PEEK or another temperature-resistant thermoplastic.

18. The coil according to claim 1, wherein the biocompatible material has a Shore hardness A of 20 to 50, preferably of 20 to 40.

19. The coil according to claim 1, wherein the coil with sheath has a thickness of 10 mm, preferably of 5-8 mm.

20. The coil according to claim 16, wherein the sheath comprises a fastening apparatus.

21. The coil according to claim 20, wherein the fastening apparatus is an eye or a sterile band.

22. The coil according to claim 1, wherein the coil has a ferromagnetic plate.

23. The coil according to claim 1, wherein the coil has two taps, one or no taps.

24. The coil according to claim 1, wherein the conductor is an HF litz wire.

25. Pair of coils for inductive transcutaneous transfer of power for the energy supply of active medical implants having an implantable secondary coil and an external primary coil, wherein one of the coils, in particular the implantable secondary coil is a coil according to claim 1.

26. The pair of coils according to claim 25, wherein the pair of coils has an alignment aid.

27. The pair of coils according to claim 26, wherein the alignment aid is a pair of magnetic disks integrated into the pair of coils, in particular a pair of ferrite magnetic disks.

28. The pair of coils according to claim 26, wherein the alignment aid is an electronic feedback with navigation aid.

29. The pair of coils according to claim 25, wherein the pair of coils comprises safety electronics.

30. The pair of coils according to claim 29, wherein the safety electronics comprises a foreign object detection and/or an analogue test inquiry and/or a digital identification and/or an overcurrent protection and/or a voltage clamp and/or a temperature detection and/or a high voltage surge stopper and/or an ESD protection and/or switch-off possibility and/or a stand-by mode of the active synchronous rectification.

31. The pair of coils according to claim 25, wherein the pair of coils transfers powers in the order of magnitude of 5-50 Watts, in particular 5-40 Watts, in particular 10-30 Watts.

32. The pair of coils according to claim 25, wherein the pair of coils is suitable for a transmission over a distance of 5-40 mm, in particular of 5-15 mm, in particular of 10-15 mm.

33. Coil system comprising a pair of coils according to claim 25, wherein it comprises a further pair of coils.

34. Coil system comprising a pair of coils according to claim 25, wherein it comprises a second implantable secondary coil.

35. Active medical implant, in particular cardiac support system, in particular having a power requirement of 5-50 Watt comprising a coil according to claim 1 or a pair of coils or a coil system.

36. Method for fabricating a coil according to claim 1.
